(19) United States
(12) Patent Application Publication
(10) Pub. No.: US 2011/0240035 A1
(43) Pub. Date: Oct. 6, 2011

(54) RESPIRATORY ASSISTANCE DEVICE AND SYSTEM AND METHOD

(75) Inventor: Mark Gillies, New South Wales (AU)

(73) Assignee: BIDIBOTS PTY LTD, Reservoir, Victoria (AU)

(21) Appl. No.: 13/130,694

(22) PCT Filed: Nov. 23, 2009
(86) PCT No.: PCT/AU2009/001519

§ 371 Date: May 23, 2011

(30) Foreign Application Priority Data
Nov. 21, 2008 (AU) 2008906036

(51) Int. Cl. A61M 16/00

(52) U.S. Cl. 128/207.18

(57) ABSTRACT

A pressurized air delivery device, system and method provide respiratory assistance to individuals, particularly infant humans. The device having nasal conduits or prongs which are in fluid communication with respective supply conduits separately supply each nasal conduit with a breathable gas such as air or oxygen. The size of the nasal conduits or prongs may be chosen according to the size of the individual's nares and the spacing between nasal conduits or prongs may be adjustable.

### FIELD OF THE INVENTION

THIS INVENTION relates to delivery of continuous positive airway pressure to an individual, such as an infant, to thereby provide respiratory assistance.

### BACKGROUND

Newborn, infants particularly those born premature often have a need for respiratory support, ranging from increased ambient oxygen, through continuous positive airways pressure (CPAP) and endotracheal ventilation to, rarely, extracorporeal membrane oxygenation (ECMO). Infants born before 30 weeks of completed gestation almost invariably need a period of CPAP or ventilation to survive.

The commonest reason for premature newborn infants to require respiratory support is due to a lack of surfactant, a protein which helps to keep the smallest of the lungs air spaces open. Treatment for this disease process has consisted of CPAP since it was realised in the mid 1970's that CPAP treatment greatly improved infants rates of survival.

Whilst replacement of surfactant via an endotracheal tube has been available in Australia since the early 1990's, this requires infants to be intubated and ventilated. However intubation and ventilation, itself, can damage infants' lungs and there has been a shift in the last 5-10 years towards CPAP, both as a modality of support post extubation and a primary support in its own right, for the majority of infants with respiratory distress.

CPAP may be delivered via a multitude of machine—patient interfaces. The best devices utilise “short binasal” prongs designed to snugly fit into the nares of infants, thereby allowing the pressure to be delivered to the infant whilst minimising the resistance of the device to the infants' work of breathing.

Current “short binasal” prongs are designed with a connection between the two prongs which insert into the infants nose. This connection may cause pressure an the nasal septum, which in extreme cases can lead to necrosis and require plastic surgery to correct. This problem is exacerbated by the mechanism by which these prongs are fixed to the infants head and face by means of hats, bonnets or headbands and the relatively bulky tubing and prongs in use.

Although the currently available devices come in a variety of sizes, both for prong size and separation between the prongs, each prong set is typically of a fixed prong size and separation.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY

The invention is broadly directed to providing respiratory assistance to an individual, typically an infant, by way of a device, system and/or method that delivers pressurized air to the nares of the individual, to alleviate one or more of the aforementioned deficiencies of the prior art.

In one aspect, the invention provides a pressurized air delivery device for providing respiratory assistance to an individual, said device comprising a plurality of nasal conduits in fluid communication with respective supply conduits connectable to a source of pressurized, breathable gas.

In another aspect, the invention provides a pressurized air delivery system for providing respiratory assistance to an individual, said system comprising a plurality of flexible nasal conduits in fluid communication with respective supply conduits connected to a source of pressurized, breathable gas.

In yet another aspect, the invention provides a method of providing respiratory assistance to an individual, said method including the step of delivering pressurized air to the nose of said individual through a plurality of nasal conduits in fluid communication with respective supply conduits connected to a source of pressurized, breathable gas.

Preferably, according to the aforementioned aspects, the individual is an infant human.

Suitably, continuous positive airway pressure is delivered to an individual by way of a plurality of conduits respectively locatable in the nares of an individual, without engaging, bearing against or otherwise contacting the nasal septum. Advantageously, continuous positive airway pressure is delivered to the individual without providing a hat, bonnet, headband or other separate means for attachment to the individual.

In a preferred embodiment, the nasal conduits are of a size adapted to fit the nares of the individual. In a particularly preferred embodiment the nasal conduits are interchangeable in size.

Preferably the nasal conduits are formed of a material that is more rigid than the supply conduits.

In certain embodiments, the nasal conduits may each comprise an inlet and an outlet, said outlet facilitating exhaust of exhaled gas to atmosphere.

Preferably, the nasal conduits further comprise respective valves to control oxygen flow to the user.

Suitably, the device and/or system further comprises a spacer to maintain a desired spacing or distance between the nasal conduits.

Preferably, the spacer is adjustable in order to select a desired spacing or distance between the nasal conduits.

In one preferred embodiment, said respective nasal prongs are maintained at a desired distance apart by an adjustable spacer that comprises a sliding rod or a band that comprises teeth or serrations. Typically, the adjustable spacer comprises an adjustment member, such as a lever, ratchet, turnbuckle, or cam, although without limitation thereto.

In a particularly preferred embodiment, the spaces comprises a band comprising teeth, ridges or serrations releasably engageable by a cam or lever.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE DRAWINGS

A preferred embodiment of the invention will now be described by way of example only, with reference to the accompanying figures in which:

FIG. 1 shows an embodiment where nasal prongs being held at a desired distance apart by rare earth magnets;

FIG. 2 shows an embodiment where nasal prongs are held at a desired distance apart by a ratchet mechanism;

FIG. 3 shows an embodiment where nasal prongs are held at a desired distance apart by a turnbuckle system;

FIG. 4 shows an embodiment where nasal prongs are spaced apart by a sliding rod mechanism;

FIG. 5 shows side and top views of different sized nasal prongs spaced apart by a sliding rod mechanism;

FIG. 6 shows side and top views of different sized prongs spaced apart by a sliding locking flat band mechanism;

FIG. 7 shows nasal prongs spaced apart by a sliding locking flat band mechanism that is adjustable by a cam mechanism;

FIG. 8 shows an example of the attachment of nasal prongs to an air supply conduit; and

FIG. 9 shows a nasal prong comprising and inlet and outlet for exhalation.

### DETAILED DESCRIPTION

Referring to FIG. 1, device 10 comprises respective nasal conduits 20A, 20B in fluid communication with respective air and/or oxygen supply conduits 30A, 30B. Nasal conduits 20A, 20B respectively comprise arms 21A, 21B normal to air supply conduits 30A, 30B and elbows 22A, 22B which respectively terminate in nasal prongs 23A, 23B. Preferably nasal conduits 20A, 20B are formed of a material that is more rigid (e.g. polymethylmethacrylate or polycarbonate) than the supply conduits which are of a relatively flexible tubing material (e.g. polyurethane).

In use, air and/or oxygen supply conduits 30A, 30B are connected to a source of pressurized air, oxygen or other gas suitable for assisting human respiration (not shown). The direction of pressurized air flow from the source is indicated by solid arrows.

According to this embodiment, spacer 40 comprises magnetic poles 41A, 41B on bridge 42, which respectively interact with magnetic poles 24A, 24B on nasal prongs 23A, 23B to maintain nasal prongs 23A, 23B at a desired separation. Typically, although not exclusively, magnetic poles 41A, 41B, 24A and 24B are produced by rare earth magnets (e.g. lanthanides such as neodymium or samarium, although without limitation thereto) located on the outside of the nares.

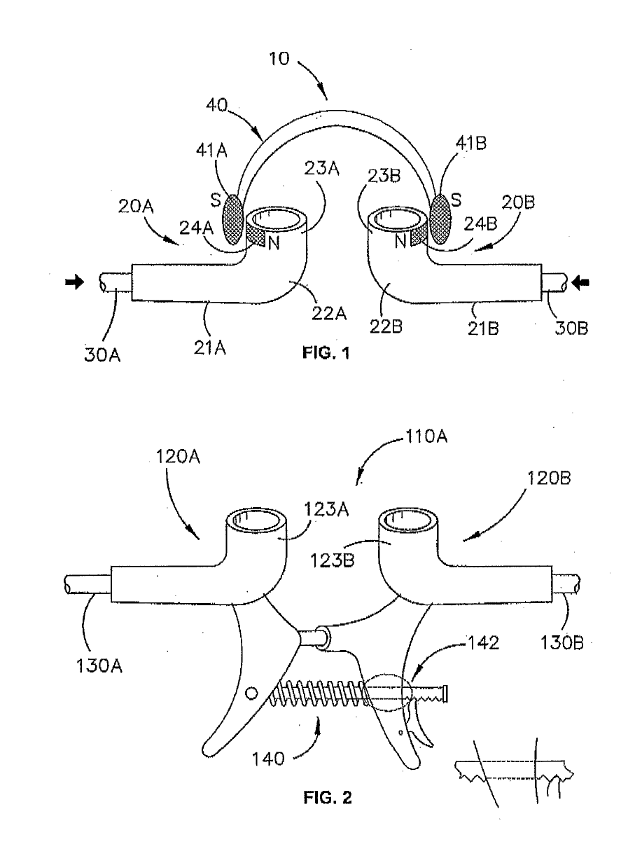

Referring to an embodiment shown in FIG. 2, device 110 comprises spacer 140 comprising adjustable ratchet 142 which maintains nasal prongs 123A, 123B at a desired separation.

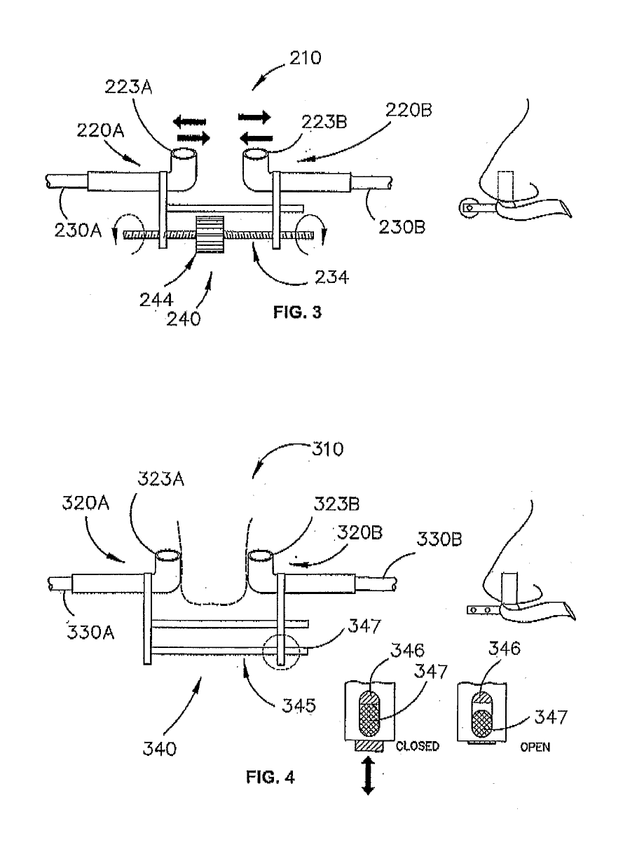

An embodiment shown in FIG. 3 shows nasal prongs 223A, 223B adjustably movable (indicated by solid horizontal arrows) to a desired distance apart by spacer 240 comprising turnbuckle 243 that is adjustable by winding wheel 244 in either direction (indicated by solid arrows).

In another embodiment of device 310 shown in FIG. 4, nasal prongs 323A, 323B are held at a desired distance apart by spacer 340 that is adjustable by way of sliding rod mechanism 345 that is adjustable by pushing button 346 to release sliding rod 347 and thereby finding the correct distance between nasal prongs 323A, 323B.

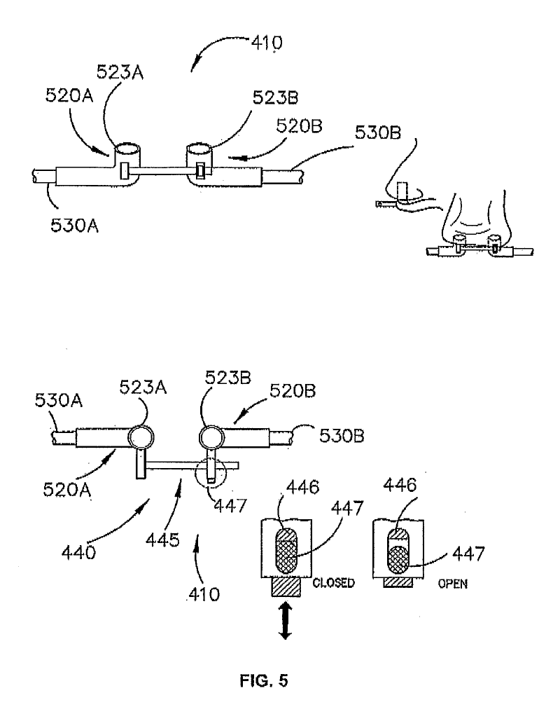

Referring now to FIG. 5, an embodiment of device 410 is shown similar to that shown in FIG. 4, wherein nasal prongs 423A, 423B are of reduced diameter compared to those in FIG. 4, demonstrating that nasal prongs 23A, 23B may be of varying, interchangeable sizes.

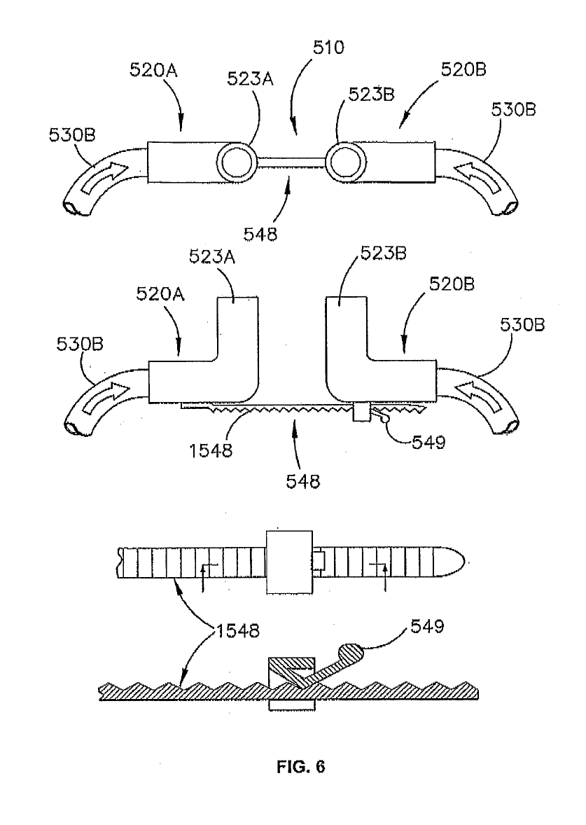

FIG. 6 shows an embodiment where nasal prongs 523A, 523B are held at a desired distance apart by spacer 540 comprising slidable band 548 that comprises teeth, serrations or ridges 1548 releasably engageable by lever 549. Lever 549 engages teeth, serrations or ridges 1548 of flat band 548 to releasably maintain the desired distance between prongs 523A, 523B and thereby allow adjustment of the spacing between prongs 523A, 523B. Preferably, slidable band 548 is located underneath arms 521A, 521B of nasal conduits 520A, 520B (relative to an individual's nares) to maximize the distance between slidable band 548 and the individual's nasal septum. This should eliminate or at least minimize any potential contact between slidable flat band 548 and the individual's nasal septum.

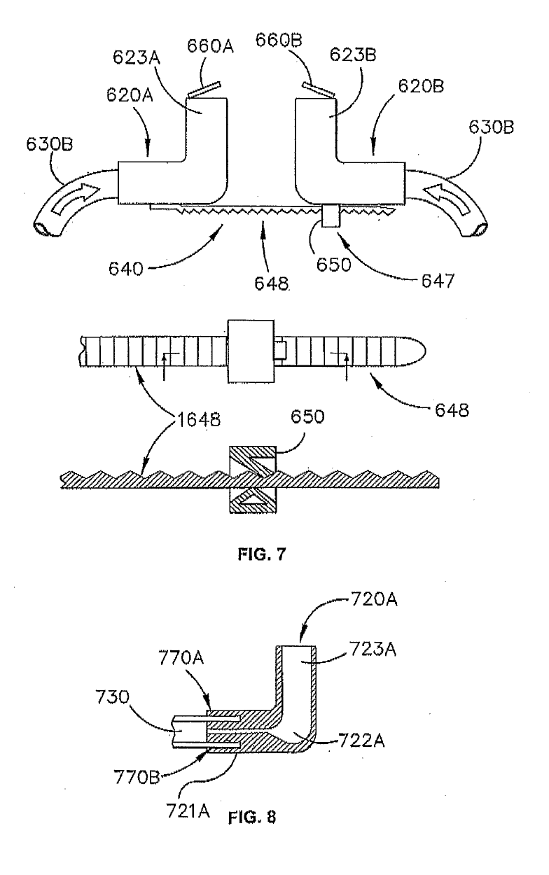

In an embodiment shown in FIG. 7, nasal prongs 623A, 623B are held at an appropriate distance apart by spacer 640 comprising slidable t band 648 that comprises teeth, serrations or ridges 1648 releasably engageable by cam 650. Cam 650 releasably engages teeth, serrations or ridges 1648 of slidable band 648 to releasably maintain the desired distance between prongs 623A, 623B and thereby allow adjustment of the spacing between prongs 623A, 623B. As in the embodiment described in FIG. 6, slidable band 648 is ideally located underneath arms 621A, 621B of nasal conduits 620A, 620B (relative to an individual's nares) to maximize the distance between slidable band 648 and the individual's nasal septum.

It should also be appreciated that slidable band 540, 640 is shown as a “flat” band in FIGS. 6 and 7. However, in certain embodiments slidable band 540, 640 may be curved to facilitate a more comfortable fit on the individual.

FIG. 8 shows an example of one way in which nasal conduit 720A is attached to air supply conduit 730A. In this example, arm 721A is pushed onto air supply conduit 730A and barbs 770A, 770B are employed to hold arm 721A in place on air supply conduit 730A.

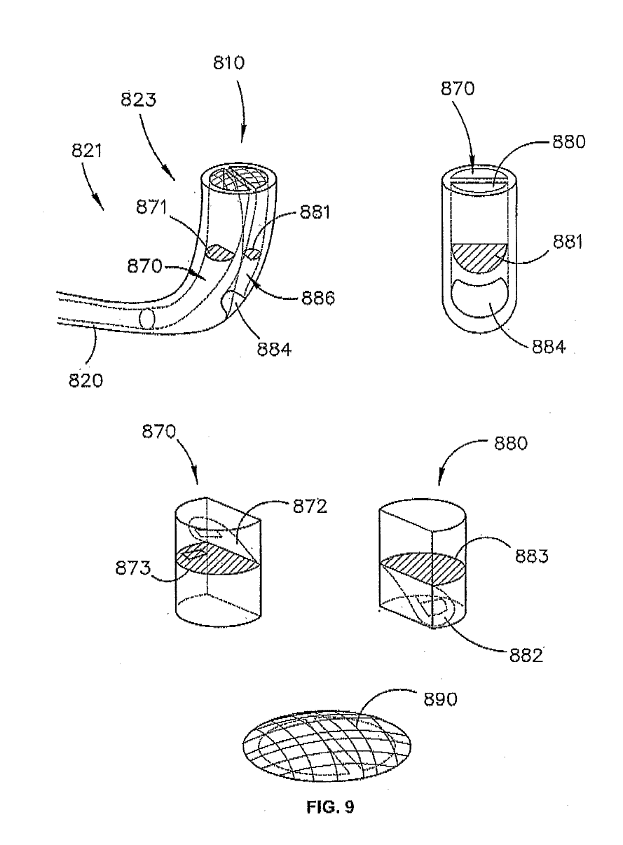

Referring to FIG. 9, there is shown an embodiment wherein device 810 comprises nasal prong 823 having inlet 870 and outlet 880, which each operate as “one-way” valves. Inlet 870 comprises leaflet 871 which moves between open position 872 and closed position 873 to enable a one-way flow of air to the nares. Outlet 880 comprises leaflet 881 which moves between closed position 882 and open position 883 to enable a one-way flow of exhaled gas from the nares to atmosphere via aperture 884. Screen 890 may also be provided to prevent accidental inhalation of leaflets 871 and/or 881.

It will be appreciated the present invention provides continuous positive airway pressure to an individual, typically an infant human, without engaging or otherwise contacting the nasal septum in a manner that causes irritation or discomfort to the user. Air, oxygen or other breathable gas is supplied via separate air supply conduits and nasal conduits, rather than via a common air supply conduit into separate nasal conduits. This eliminates the portion of the common air supply conduit that contacts and irritates the nasal septum. Furthermore, the nasal prongs may be provided in a variety of interchangeably different sizes to suit the size of the nares of the user, which also assists the objective of reducing the level of irritation and discomfort.

Another advantage of the present invention is that the device needs limited fixation, (even just a piece of tape may suffice) rather than the bonnets, hats, head-bands etc. required with prior art devices. Furthermore, many prior art devices require significant nursing expertise to attach and supervise in use. The present invention can be fitted and supervised by any person familiar with standard nasal cannulae.

In summary, the present invention provides improved nasal septal protection, ease of attachment, ease of nursing care, reduced weight and bulk, the ability to adapt nasal conduit size to different nare sizes (sometimes if a child has been nasally intubated, one nare is larger than the other), and variable separation between respective conduits.

Throughout this specification, the aim has been to describe the preferred embodiments of the invention without limiting the invention to any one embodiment or specific collection of features. Various changes and modifications may be made to the embodiments described and illustrated without departing from the present invention.

1. A pressurized air delivery device for providing respiratory assistance to an individual, said device comprising a plurality of nasal conduits in fluid communication with respective supply conduits connectable to a source of pressurized, breathable gas.

2. A pressurized air delivery system for providing respiratory assistance to an individual, said system comprising a plurality of flexible nasal conduits in fluid communication with respective supply conduits connected to a source of pressurized, breathable gas.

3. The device of claim 1, further comprising a spacer to maintain a desired spacing or distance between the nasal conduits.

4. The device of claim 3, wherein the spacer is adjustable in order to select a desired spacing or distance between the nasal conduits.

5. The device of claim 4, wherein said adjustable spacer comprises an adjustment member.

6. The device of claim 4, wherein the adjustment member comprises a lever, ratchet or cam.

7. The device, of claim 1 wherein the nasal conduits are formed of a material that is more rigid than the supply conduits.

8. The device of claim 1, wherein the nasal conduits are of interchangeable sizes.

9. The device, of claim 1, wherein the nasal conduits each further comprise a valve.

10. The device of claim 1, wherein each said nasal conduit comprises an inlet and an outlet, said outlet facilitating exhaust of exhaled gas to atmosphere.

11. The device of claim 1, wherein the breathable gas is air or oxygen.

12. A method of providing respiratory assistance to an individual, said method including the step of delivering pressurized air to the nose of said individual through a plurality of nasal conduits in fluid communication with respective conduits connected to a source of pressurized, breathable gas.

13. The method of claim 12, wherein the individual is an infant human.

14. The method of claim 12, wherein continuous positive airway pressure is delivered to an individual by way of a plurality of conduits respectively locatable in the nares of an individual, without engaging or otherwise contacting the nasal septum.

15. The method of claim 12, using a pressurized air delivery device for providing respiratory assistance to an individual, said device comprising a plurality of nasal conduits in fluid communication with respective supply conduits connectable to a source of pressurized, breathable gas.

16. The method of claim 13, using a pressurized air delivery device for providing respiratory assistance to an individual, said device comprising a plurality of nasal conduits in fluid communication with respective supply conduits connectable to a source of pressurized, breathable gas.

17. The method of claim 14, using a pressurized air delivery device for providing respiratory assistance to an individual, said device comprising a plurality of nasal conduits in fluid communication with respective supply conduits connectable to a source of pressurized, breathable gas.

18. The system of claim 2 further comprising an adjustable spacer in order to select a desired spacing or distance between the nasal conduits.
